(19) United States
(12) Patent Application Publication
(10) Pub. No.: US 2021/0393257 A1
(43) Pub. Date: Dec. 23, 2021

(54) CLAMP INCLUDING APERTURE

(71) Applicant: Max MICHALSKI, Boston, MA (US)

(72) Inventor: Max MICHALSKI, Boston, MA (US)

(21) Appl. No.: 17/129,005

(22) Filed: Dec. 21, 2020

Related U.S. Application Data
(60) Provisional application No. 63/042,903, filed on Jun. 23, 2020.

(51) Int. Cl. A61B 17/04

(52) U.S. Cl. CPC A61B 17/0483

(57) ABSTRACT

A clamp including a first aperture is provided. In some examples, the clamp includes a first handle portion. A first arm may be connected to the first handle portion, where the first arm includes a first portion of the first aperture. The clamp may further include a second handle portion. A second arm may be connected to the second handle portion, where the second arm includes a second portion of the first aperture.

### CROSS REFERENCE TO RELATED APPLICATIONS

This application claims the benefit of U.S. Provisional Application No. 63/042,903, entitled “CLAMP INCLUDING APERTURE,” filed Jun. 23, 2020, the content of which is hereby incorporated by reference in its entirety for all purposes.

### FIELD

This application relates generally to a clamp including an aperture, and specifically to a clamp including an aperture for object passage.

### BACKGROUND

Clamps, such as tissue clamps, are beneficial for many purposes, including positioning or affixing various objects during medical procedures. However, existing clamps are not well equipped to handle certain tasks, such as delivering sutures through tendons. For example, conventional clamps do not provide functionality to deliver a suture through a tendon with precision, reliability, and accuracy. Thus, an improved clamp having an aperture for object passage is desired.

### SUMMARY

An apparatus and method for using a clamp is provided. In some examples, the clamp includes a first handle portion. A first arm may be connected to the first handle portion, wherein the first arm includes a first portion of a first aperture. The clamp may further include a second handle portion. A second arm may be connected to the second handle portion, wherein the second arm includes a second portion of the first aperture.

In some examples, the clamp may be opened by moving a first handle portion of the clamp and a second handle portion of the clamp. A first object may be positioned within a first portion of an aperture, wherein the first portion of the aperture is included in a first arm connected to the first handle portion. The first portion of the aperture may contact a first portion of the object. The clamp may be closed in order to position the object within a second portion of the aperture, wherein the second portion of the aperture is included in a second arm connected to the second handle portion. The second portion of the aperture may contact a second portion of the object opposite to the first portion of the object. A second object may be delivered through the aperture and through the first object, wherein the second object is moved through the aperture and through the first object in a first direction. By delivering the second object in the first direction, the second object may be removed from the aperture and from the first object. The second object may be coupled to a thread. The thread may be further removed from the second object in order to couple the thread to the first object.

### BRIEF DESCRIPTION OF THE DRAWINGS

FIG. 1A illustrates an exemplary clamp in a closed position including one aperture.

FIG. 1B illustrates an exemplary clamp including a plurality of apertures.

FIG. 1C illustrates an exemplary clamp in an open position including one aperture.

FIGS. 2A-2C illustrate an exemplary process for using a clamp including an aperture.

FIGS. 3A-3B illustrate an exemplary clamp including an aperture with a plurality of teeth.

### DETAILED DESCRIPTION

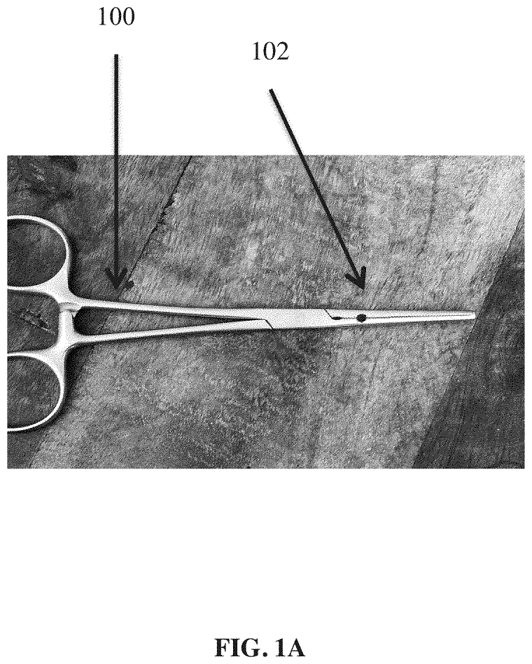

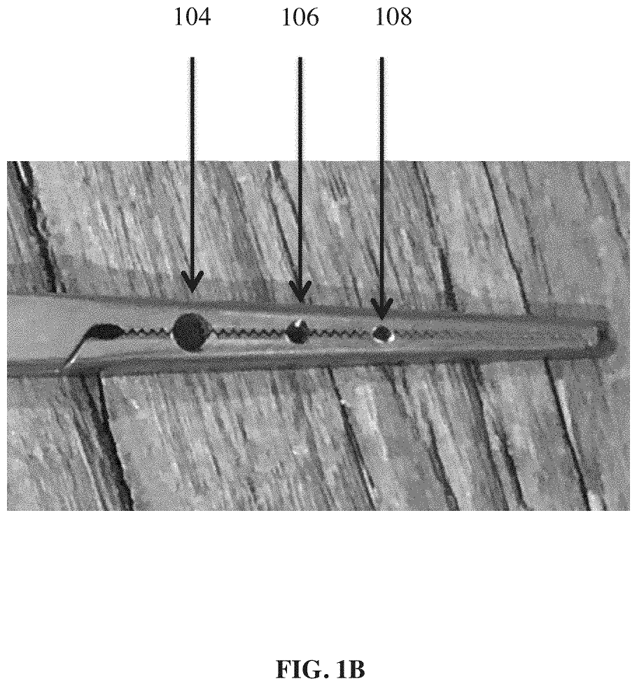

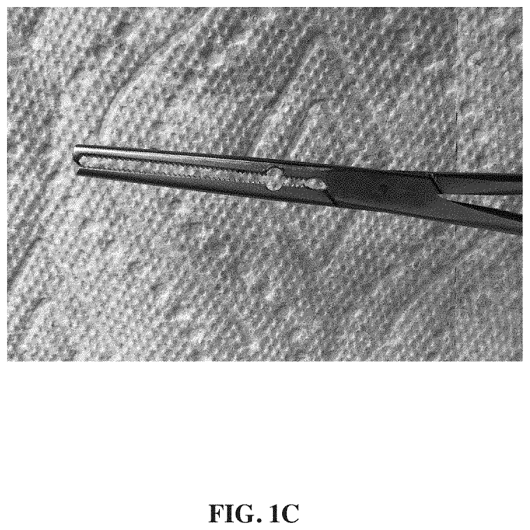

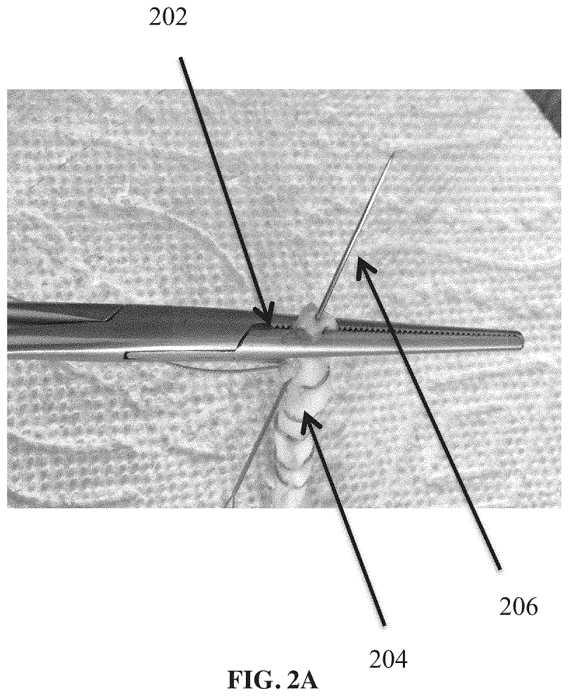

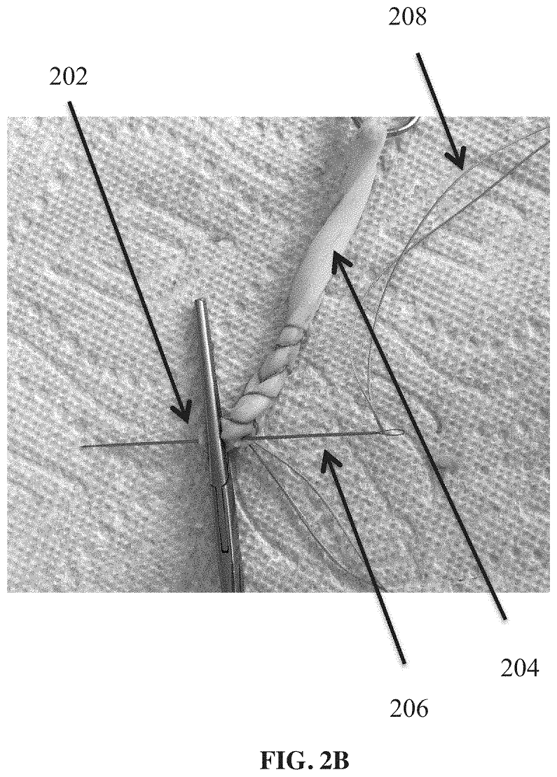

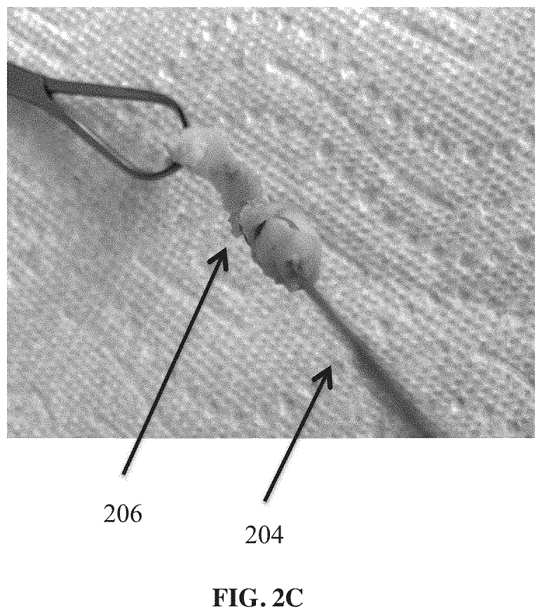

FIGS. 1A-1C illustrate an exemplary clamp. As shown in FIG. 1A, clamp 100 may include an aperture 102. Aperture 102 may have a predetermined size and shape, such as a diameter of 1-millimeter and a circular shape. Clamp 100 may also include multiple apertures. For example, referring now to FIG. 1B, clamp 100 may include apertures 104-108. The multiple apertures may each be associated with predetermined sizes and shapes. For example, each aperture may have a circular shape. Aperture 104 may have a larger size than aperture 106, where aperture 106 may have a larger size than aperture 108. The aperture(s) may be centered along the clamp arms, such that each arm includes exactly or approximately one half of the aperture. For example, one arm may include a cavity in the shape of a half-circle and the other arm may include a cavity in the shape of a half-circle. As shown in FIG. 1C, opening the clamp results in the aperture becoming separated in order to allow a user to easily affix an item within the clamp, as discussed with respect to FIGS. 2A-2C.

FIGS. 2A-2C illustrate a method of using a clamp including an aperture, such as claim 100. In some examples, the clamp may be utilized in order to improve delivery of a needle and/or suture through various objects, such as a tendon. As shown in FIG. 2A, an object may be positioned within the aperture. For example, a user of the clamp may open the clamp (as depicted in FIG. 1C), and insert or otherwise position the object within the aperture. Once the objected is positioned within the aperture, the user may close the clamp to firmly maintain the objection position. As shown in FIG. 2A, a tendon 204 may be positioned within aperture 202. Upon placement of tendon 204 within aperture 202 and closing of the clamp, a needle 206 may be driven through tendon 204. In particular, the needle 206 may be driven through aperture 202 which includes tendon 204.

FIG. 2B illustrates an alternate view of the clamp including the aperture 202 with tendon 204 and needle 206. Additionally depicted in FIG. 2B is a suture 208 affixed to one end of needle 206. In some examples, upon driving the needle 206 through the tendon 204, a user may pull the needle 206 through aperture 202 and thus pull the needle 206 through tendon 204. With reference to FIG. 2B, for example, the user may pull needle 206 towards the left in order to pull the needle 206 completely through tendon 204. After pulling needle 206 through tendon 204, the user may continually pull the needle 206 such that suture 208 moves through aperture 202 and thus through tendon 204. After completely pulling suture 208 through tendon 204, the user may open the clamp to release tendon 204 from the clamp. For example, as depicted in FIG. 2C, following the release of tendon 204 from the clamp, suture 208 may pass directly or substantially directly through the center of tendon 204. In this manner, suture 208 may be firmly coupled to tendon 204.

In some examples, the aperture(s) may have varying diameters along the depth of the aperture. In general, the varying diameters may cause the tendon to have a “cone” shaped end when the tendon is compressed within the aperture. Specifically, for example, aperture 202 may have a smaller diameter (e.g., 3 mm) on one side of the clamp arms (e.g., the top side of the clamp side visible in FIG. 2A). Aperture 202 may have a larger diameter (e.g., 5 mm) on the other side of the clamp arms (e.g., the bottom side of the clamp not visible in FIG. 2A). The portion of the aperture between the top side of the clamp arms and the bottom side of the clamp arms may have a gradually increasing diameter moving from the top side of the clamp arms to the bottom side of the clamp arms. For instance, the middle section of the aperture may have a diameter that is approximately sized halfway between the sizes of the smaller and larger diameter, respectively (e.g., 4 mm). In some examples, the diameter of middle section of the aperture may be smaller or larger than the halfway-between size in order to allow for various aperture shapes.

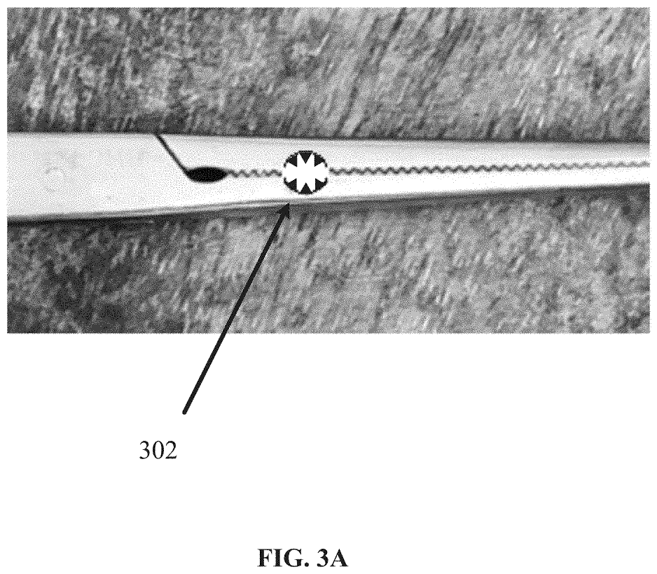

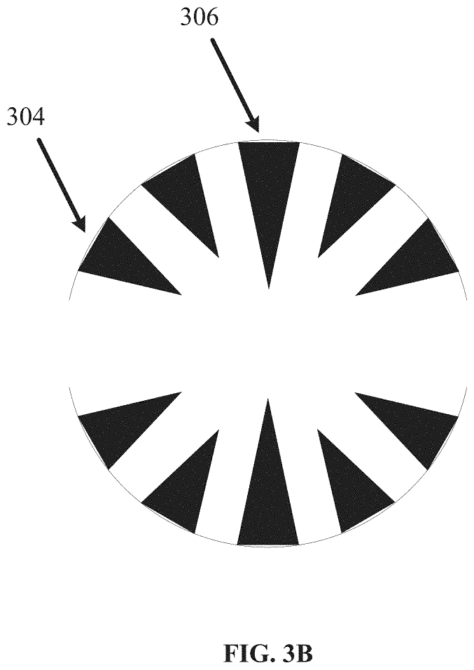

FIG. 3A illustrates a clamp in a closed position including an aperture with a plurality of teeth. In particular, each tooth of the plurality of teeth may assist a user in grasping an object when using the clamp. FIG. 3B illustrates a close-up view of an aperture including a plurality of teeth, wherein the aperture corresponds to a clamp in a partially open position. For example, the aperture may include teeth 304 and 306. Each tooth may be angled towards the center of the aperture in order to deliver a maximum contact area for penetration of an object being grasped by the camp (e.g., a tendon). In some examples, various teeth of the plurality of teeth may be associated with varying sizes. As shown in FIG. 3B, tooth 306 may be larger than tooth 304, for example. In particular, tooth 306 may include a longer length than tooth 304. In some examples, tooth 306 may generally be associated with a larger diameter than tooth 304. Various teeth may also may shaped differently. For example, one or more teeth may include multiple pointed ends, and/or may include curved ends (e.g., hooks).

Various descriptions and characterizations included herein are not intended to be limiting to the clamp including the aperture. The illustrations and associated descriptions are also not intended to be exhaustive or to limit the claimed invention to any specific form. Those of skill in the art will appreciate that changes and modifications to the clamp may become apparent. Those changes are included within the scope of this disclosure.

In general, a clamp is provided. In some examples, the clamp includes a first handle portion and a first arm connected to the first handle portion, wherein the first arm includes a first portion of a first aperture. In some examples, the clamp includes a second handle portion and a second arm connected to the second handle portion, wherein the second arm includes a second portion of the first aperture. In some examples, the first aperture has a circular shape. In some examples, the first arm includes a first portion of a second aperture, and the second arm includes a second portion of the second aperture. In some examples, the first arm includes a first portion of a third aperture, and the second arm includes a second portion of the third aperture.

In some examples, the first portion of the first aperture, the second portion of the first aperture, and the third portion of the first aperture are arranged along an axis, wherein the axis is parallel to a direction of the first arm. In some examples, the first portion of the second aperture, the second portion of the second aperture, and the third portion of the second aperture are arranged along an axis, wherein the axis is parallel to a direction of the second arm. In some examples, the first portion of the aperture includes a first plurality of teeth and the second portion of the aperture includes a second plurality of teeth. In some examples, the first plurality of teeth includes a first tooth associated with a first length and a second tooth associated with a second length, wherein the first length is longer than the second length. In some examples, the first object corresponds to a tendon and the second objet correspond to a needle. In some examples, the first aperture comprises a first end and a second end, wherein the first end is associated with a first diameter and the second end is associated with a second diameter larger than the first diameter.

In some examples, a method of using a clamp including an aperture is provided. In some examples, the clamp may be opened by moving a first handle portion of the clamp and a second handle portion of the clamp. In some examples, a first object may be positioned within a first portion of an aperture, wherein the first portion of the aperture is included in a first arm connected to the first handle portion, and wherein the first portion of the aperture contacts a first portion of the object. In some examples, the clamp may be closed in order to position the object within a second portion of the aperture, wherein the second portion of the aperture is included in a second arm connected to the second handle portion. In some examples, the second portion of the aperture contacts a second portion of the object opposite to the first portion of the object. In some examples, a second object is delivered through the aperture and through the first object, wherein the second object is moved through the aperture and through the first object in a first direction. In some examples, the second object may be removed, by delivering in the first direction, the second object from the aperture and from the first object, wherein the second object is coupled to a thread. In some examples, the thread may be removed from the second object in order to couple the thread to the first object.

In some examples, positioning a first object within a first portion of an aperture includes attaching the first object to a first plurality of teeth included in the first portion of the aperture. In some examples, attaching the first object to a first plurality of teeth included in the first portion of the aperture includes piercing a first depth of the first object with the first plurality of teeth, wherein the first portion of the aperture corresponds to a first half circle of the aperture. In some examples, attaching the first object to a first plurality of teeth included in the first portion of the aperture includes piercing a first depth of the first object with at least one first tooth of the first plurality of teeth and a second depth of the first object with at least one second tooth of the first plurality of teeth, wherein the first depth is less than the second depth, and the at least one first tooth is shorter than the at least one second tooth.

In some examples, closing the clamp in order to position the first object within a second portion of the aperture includes attaching the first object to a second plurality of teeth included in the second portion of the aperture. In some examples, attaching the first object to a second plurality of teeth included in the second portion of the aperture includes piercing a second depth of the first object with the second plurality of teeth, wherein the second portion of the aperture corresponds to a second half circle of the aperture. In some examples, attaching the first object to a second plurality of teeth included in the second portion of the aperture includes piercing a first depth of the first object with at least one first tooth of the second plurality of teeth, piercing a second depth of the first object with at least one second tooth of the second plurality of teeth, wherein the first depth is less than the second depth, and the at least one first tooth is shorter than the at least one second tooth. In some examples, delivering a second object through the aperture and through the first object includes delivering the second object through a center portion of the first object. In some examples, delivering a second object through the aperture and through the first object includes delivering the thread through a center portion of the first object. In some examples, the clamp is opened before removing the thread from the second object in order to couple the thread to the first object.

What is claimed is:

1. A clamp, comprising:
a first handle portion;
a first arm connected to the first handle portion, wherein the first arm includes a first portion of a first aperture;
a second handle portion; and
a second arm connected to the second handle portion, wherein the second arm includes a second portion of the first aperture.

2. The clamp of claim 1, wherein the first aperture has a circular shape.

3. The clamp of claim 1, wherein
the first arm includes a first portion of a second aperture, and
the second arm includes a second portion of the second aperture.

4. The clamp of claim 1, wherein
the first arm includes a first portion of a third aperture, and
the second arm includes a second portion of the third aperture.

5. The clamp of claim 4, wherein the first portion of the first aperture, the first portion of the second aperture, and the first portion of the third aperture are arranged along an axis, wherein the axis is parallel to a direction of the first arm.

6. The clamp of claim 4, wherein the second portion of the first aperture, the second portion of the second aperture, and the second portion of the third aperture are arranged along an axis, wherein the axis is parallel to a direction of the second arm.

7. The clamp of claim 1, wherein
the first portion of the first aperture includes a first plurality of teeth, and
the second portion of the first aperture includes a second plurality of teeth.

8. The clamp of claim 7, wherein,
the first plurality of teeth comprises:
a first tooth associated with a first length, and
a second tooth associated with a second length, wherein the first length is longer than the second length.

9. The clamp of claim 1, wherein the first object corresponds to a tendon and the second object corresponds to a needle.

10. The clamp of claim 1, wherein the first aperture comprises a first end and a second end, and wherein
the first end is associated with a first diameter, and
the second end is associated with a second diameter larger than the first diameter.

11. A method of using a clamp including an aperture, the method comprising:
opening the clamp by moving a first handle portion of the clamp and a second handle portion of the clamp;
positioning a first object within a first portion of an aperture, wherein the first portion of the aperture is included in a first arm connected to the first handle portion, and wherein the first portion of the aperture contacts a first portion of the first object;
closing the clamp in order to position the first object within a second portion of the aperture, wherein the second portion of the aperture is included in a second arm connected to the second handle portion, and wherein the second portion of the aperture contacts a second portion of the first object opposite to the first portion of the first object;
delivering a second object through the aperture and through the first object, wherein the second object is moved through the aperture and through the first object in a first direction;
removing, by delivering the second object in the first direction, the second object from the aperture and from the first object, wherein the second object is coupled to a thread; and
removing the thread from the second object in order to couple the thread to the first object.

12. The method of claim 11, wherein positioning a first object within a first portion of an aperture comprises:
attaching the first object to a first plurality of teeth included in the first portion of the aperture.

13. The method of claim 12, wherein attaching the first object to a first plurality of teeth included in the first portion of the aperture comprises:
piercing a first depth of the first object with the first plurality of teeth, wherein the first portion of the aperture corresponds to a first half circle of the aperture.

14. The method of claim 13, wherein attaching the first object to a first plurality of teeth included in the first portion of the aperture comprises:
piercing a first depth of the first object with at least one first tooth of the first plurality of teeth;
piercing a second depth of the first object with at least one second tooth of the first plurality of teeth, wherein
the first depth is less than the second depth, and
the at least one first tooth is shorter than the at least one second tooth.

15. The method of claim 11, wherein closing the clamp in order to position the first object within a second portion of the aperture comprises:
attaching the first object to a second plurality of teeth included in the second portion of the aperture.

16. The method of claim 15, wherein attaching the first object to a second plurality of teeth included in the second portion of the aperture comprises:
piercing a second depth of the first object with the second plurality of teeth, wherein the second portion of the aperture corresponds to a second half circle of the aperture.

17. The method of claim 16, wherein attaching the first object to a second plurality of teeth included in the second portion of the aperture comprises:
piercing a first depth of the first object with at least one first tooth of the second plurality of teeth;
piercing a second depth of the first object with at least one second tooth of the second plurality of teeth, wherein
the first depth is less than the second depth, and
the at least one first tooth is shorter than the at least one second tooth.

18. The method of claim 11, wherein delivering a second object through the aperture and through the first object comprises:
delivering the second object through a center portion of the first object.

19. The method of claim 11, wherein delivering a second object through the aperture and through the first object comprises:
delivering the thread through a center portion of the first object.

20. The method of claim 11, further comprising:
opening the clamp before removing the thread from the second object in order to couple the thread to the first object.
